(19) United States
(12) Patent Application Publication
(10) Pub. No.: US 2012/0136414 A1
(43) Pub. Date: May 31, 2012

(54) Microcurrent-Generating Topical Or Cosmetic Systems, And Methods Of Making And Using The Same

(76) Inventors: Vasile Ionita-Manzatu, Old Bethpage, NY (US); Jack Lombardi, Massapequa, NY (US)

(21) Appl. No.: 13/376,756

(22) PCT Filed: Apr. 21, 2010
(86) PCT No.: PCT/US10/31917

§ 371 Date: Feb. 3, 2012

Related U.S. Application Data
(60) Provisional application No. 61/187,098, filed on Jun. 15, 2009.

(51) Int. Cl. A61N 1/36

(52) U.S. Cl. 607/50; 607/2

(57) ABSTRACT

The present invention relates to a topical or cosmetic system that includes a metal and a semiconductor element. One element acts as an electron donor and the other element acts as an electron acceptor. Such metal and semiconductor elements are in contact through a metal/semiconductor junction. The system also includes an electrically conductive medium that extends across the skin surface and electrically connects the metal and semiconductor elements. The electrical resistance of the conductive medium is significantly smaller than that of the metal/semiconductor junction, so that the majority of electrical current generated between the metal element and the semiconductor element flows across the skin surface through the conductive medium in the absence of any power source. The present invention also relates to methods of using the above-described system for preventing or treating skin damage.

### CROSS-REFERENCE TO RELATED APPLICATIONS

The present application claims priority of U.S. Provisional Application No. 61/187,098 filed on Jun. 15, 2009.

### FIELD OF THE INVENTION

The present invention relates to novel topical or cosmetic systems and skin treatment methods. Specifically, the invention relates to topical or cosmetic systems that, when applied to a skin surface, consistently generate a mild electrical current that flows across the skin surface in a sustainable manner and in absence of any external power source. More specifically, such electrical current is characterized by a current intensity particularly effective for preventing or reducing skin damage and improving skin quality without causing any irritation or inflammation.

### BACKGROUND OF THE INVENTION

It has long been recognized that there is a normal transcutaneous electric potential associated with mammalian skin (see, e.g., Robert Edelberg, in, The Biophysical Properties of the Skin, Harry Elden (ed.), Chapter 15, Wiley Interscience, 1971). This potential is to a great extent influenced by the presence of sweat glands and hair, and thus the strength of the potential may differ both spatially and temporally on the skin. However, even in nonglandular areas of the skin, there is a fairly strong, measurable current produced across the epidermis, in essence forming an intrinsic skin battery. Although most measurements have been conducted on non-human mammals, considerable evidence indicates that the same type of battery exists on human skin as well (Barker et al., Am. J. Physiol. 242: R358-R366, 1982). Such batteries have been known to exist in amphibians, where they apparently serve a function in sodium uptake and appendage regeneration. However, their purpose in a nonaquatic vertebrate was not readily apparent. Based on observations of fairly strong voltage gradients at the margins of wounds, Barker et al. and others have suggested that in mammals the skin currents may be important in the process of wound healing.

Further evidence of the importance of electrical currents in the maintenance of healthy skin has been shown in the successful use of electrotherapy in treatment of skin damages. For example, Carley and Wainapel (Arch. Phys. Med. Rehabil. 66: 443-446, 1985) have shown that treatment of indolent ulcers with low intensity direct current significantly increased the healing rate of those treated individuals relative to individuals treated with conventional therapy, with a concomitant reduction in pain and discomfort in those treated with electrotherapy. Similarly, Grace Chao et al. (Connective Tissue Research, 48: 188, 2007) noted the effects of applied DC electric field on the ligament fibroblast migration and wound healing processes, and Alvarez O M et al. (J. Invest. Dermatol., 81(2): 144-148, 1983, August) demonstrated that the healing of superficial skin wounds was stimulated by external electrical current.

Therefore, it appears that the maintenance of an electric current on the skin is associated with the continued well-being of undamaged skin, and that application of an electrical current to damaged skin can be highly beneficial to the healing process of such damaged skin. In addition to the reported treatment of ulcers, there are a number of other skin conditions involving irritation or inflammation which could also potentially benefit from preventive and/or therapeutic application of a low intensity current. However, the means for delivery of electrical current to skin reported in the medical literature typically involve the use of external power source and monitoring devices, which would be prohibitively expensive and complicated for the treatment of less serious skin damages, which are not life-threatening but nonetheless painful and irritating.

Although dermal patches featuring ultra-thin power supplies and electrodes printed or laminated onto elastic and flexible plastic substrates have become commercially available in recent years, such dermal patches are mostly designed for aiding transdermal delivery of active cosmetic or pharmaceutical ingredients into the skin. A typical dermal patch used for conventional transdermal delivery of active cosmetic or pharmaceutical ingredients into the skin contains a printed, ultra-thin micro battery connected to an anode and a cathode that are in direct contact with the skin surface. The electrical potential differences between the anode and the cathode, as provided by the micro battery, generate an electric current that flows from the anode through the skin to the cathode, which in turn asserts repulsive electromotive forces on charged active cosmetic or pharmaceutical ingredients on the skin surface, i.e., the positively charged anode will repel positively charged active cosmetic or pharmaceutical ingredients into the skin, while the negatively charged cathode will repel negatively charged active cosmetic or pharmaceutical ingredients into the skin.

However, the current intensity achieved by such conventional dermal patch is typically in the milli-ampere (mA) range, which may cause irritation or inflammation of the skin. Further, because the electric current generated by such conventional dermal patch flows through the skin, the current intensity is significantly affected by various factors, such as the pH, moisture content, and resistance of skin, which can vary widely from person to person and even for the same person at different times of the day. In addition, the conventional dermal patches still require presence of power supplies, which significantly increase the complexity and costs of manufacturing and present additional modes of failure.

In contrast with the conventional devices described hereinabove, the present invention provides a simple and novel system for consistently generating a mild electrical current that flows across the skin surface, instead of through the skin, in a sustainable manner without the need for any external power source. Inventors of the present invention have discovered that such mild cross-flow electrical current is surprising and unexpectedly effective for preventing or treating skin damage and improving skin quality, even in absence of any cosmetic or skin care actives. In U.S. patent application Ser. No. 12/388,661, incorporated herein by reference in its entirety, the inventors of the present invention disclose a system that functions similarly, but has a different construction.

### SUMMARY OF THE INVENTION

The present invention in one aspect relates to a topical or cosmetic system for preventing or treating skin damage, comprising:
(a) a first element comprising at least one elemental metal;
(b) a second element comprising at least one semiconductor material, wherein said first and second element are in contact with each other through a metal/semiconductor junction having a first electrical resistance; and
(c) a third element extending across the skin surface, wherein said third element comprises an electrically conductive medium for electrically connecting said first and second elements, said electrically conductive medium has a second electrical resistance, wherein at least one of the first and the second electrical resistance is less than the other of the first and the second electrical resistance, such that an electrical current is generated between the first and second element that flows across the skin surface through said conductive medium in the absence of any power source.

Another aspect of the present invention relates to a topical or cosmetic device for preventing or treating skin damage, comprising a substrate patch having at least one side with sufficient adhesiveness for application to a skin surface, wherein a first region of the substrate patch comprises at least one elemental metal and a second region of the substrate patch comprises at least one semiconductor material. The first and second regions are in contact with each other through a metal/semiconductor junction having a first electrical resistance. The substrate patch comprises an electrically conductive medium for electrically connecting the first region and the second region. The electrically conductive medium has a second electrical resistance. At least one of the first and the second electrical resistance is significantly less than the other of the first and the second electrical resistance, such that an electrical current is generated between the first and second regions that flows across the skin surface through said conductive medium of the substrate patch in the absence of any power source.

Other aspects and features of the present invention will become more apparent from the following detailed description with reference to the accompanying drawings.

### BRIEF DESCRIPTION OF THE DRAWINGS

FIG. 1 is a cross-sectional view of a topical or cosmetic device of the invention as applied to a skin surface; and

FIG. 2 is a cross-sectional view of a second embodiment of the topical or cosmetic device of the invention shown in FIG. 1;

FIG. 3 is a cross-sectional view of a third embodiment of the topical or cosmetic device of the invention shown in FIG. 1.

### DETAILED DESCRIPTION OF THE INVENTION, AND PREFERRED EMBODIMENTS THEREOF

It is to be understood that the invention is not limited to the details of construction and arrangement of specific components set forth in the following description, which is directed to preferred embodiments of the present invention and by no means should be construed as limiting the broad scope of the present invention. The present invention is applicable to, and intended to cover, other embodiments not specifically described herein, as long as such embodiments are consistent with the general principles and spirit of the present invention.

The present invention overcomes various deficiencies of prior art devices as described in the Background section, by employing a micro electrical current that flows across the skin surface, instead of through the skin, for preventing or treating skin damage and improving overall appearance of the skin. Further, the present invention provides a simple and innovative system or device for generating such a micro electrical current in a sustainable manner without the need for any external power source.

The principles and operation of the systems and devices of the present invention can be better understood with references to the exemplary embodiments illustrated in the following drawing figures.

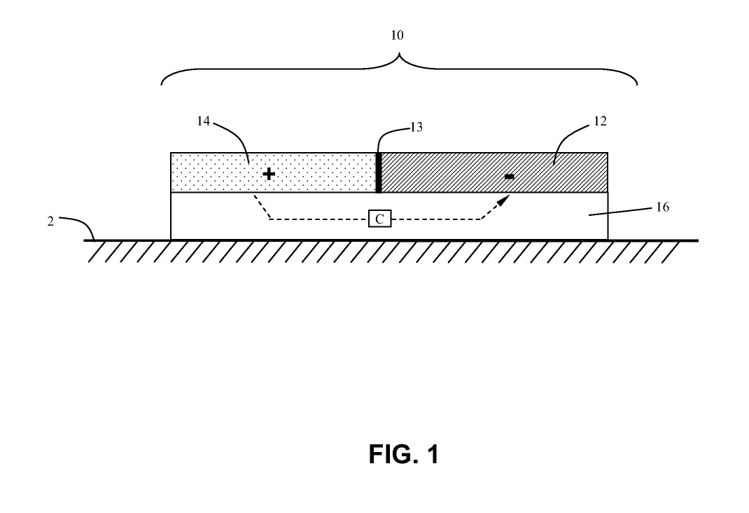

FIG. 1 shows a schematic view of a topical or cosmetic system 10 as applied to a skin surface 2, according to one embodiment of the present invention. Specifically, the topical or cosmetic system 10 includes a first element 12 comprising at least one elemental metal and a second element 14 comprising at least one semiconductor material. The first element 12 is in contact with the second element 14 through a metal/semiconductor junction 13. The metal/semiconductor junction 13 has a first electrical resistance. At least one of the first element 12 or the second element 14 is capable of acting as an electron donor (i.e., a negative electrode or a cathode) and releasing electrons, while the other of the first element 12 or the second element 14 is capable of acting as an electron acceptor (i.e., a positive electrode or an anode) and gaining the electrons released by the donor element (12 or 14) through an external circuit. For illustrative purposes, in FIG. 1, the first element 12 is designated as an electron donor (“−”) and the second element 14 is designated as an electron acceptor (“+”) but it will be understood that either element can be the donor and the other element will be the acceptor. The system 10 also includes a third element 16, which supports elements 12 and 14 and forms a contact surface for the system 10 across the skin surface 2. The third element 16 comprises an electrically conductive medium. The electrically conductive material of the third element 16 has a second electrical resistance that is less than the first electrical resistance of the junction 13. For example, it is believed that a suitable second electrical resistance would preferably be less than 20%, but more preferably less than 10%, and most preferably less than 1% of the first electrical resistance. However, it should be understood that the percent that the second electrical resistance is less than the first electrical resistance may be any percent suitable to provide through the third element 16 a current having a density or intensity ranging from about 1 μA to about 1000 μA (of course, also taking into account the electrical conductivity of the material selected for element 16).

In this manner, the third element 16 forms an external circuit that electrically connects the first and second elements 12 and 14 together. Because the second electrical resistance of the electrically conductive material of the third element 16 is significantly less than the first electrical resistance of the junction 13 an electric current “C” (as indicated by the arrow with a dotted line) is generated through the third element 16 flowing in the absence of any power source from the second element 14 to the first element 12 (or in the opposite direction if the donor/acceptor are reversed). The electric current C, which is characterized by a current intensity in the micro-ampere (μA) range, flows between the first and second elements 12 and 14 along a path that extends across the skin surface 2 (i.e., through the third element 16), instead of through the skin. Such a cross-flow microcurrent has demonstrated surprising and unexpected effectiveness in preventing or treating skin damages and improving the overall appearance of the skin. Further, because its flow path extends through the conductive medium of the third element 16 across the skin surface 2, instead of through the skin, the cross-flow microcurrent generated by the topical or cosmetic system of the present invention is significantly less affected by variations in the pH, moisture content, and resistance of the skin, in comparison with the prior art dermal patch devices described in the Background section.

As illustrated, the first element 12 may comprise any elemental metal capable of acting as an electron donor or acceptor with respect to the semiconductor material selected for the second element 14. Conversely, the second element 14 may comprise any semiconductor material suitable and capable of acting as an electron acceptor or donor with respect to the elemental metal selected for the first element 12. The first electrical resistance of the metal/semiconductor junction 13 is determined by a number of factors including, but not limited to, the size of the junction 13 (i.e., the area of contact between the first element and the second element), the elemental metal selected for the first element and the composition of the semiconductor material selected for the second element. For example, to increase the first electrical resistance of the junction 13, the size of the junction 13 can be minimized. The first electrical resistance of the junction 13 can also be increased by increasing the oxidation level of the composition of the semiconductor material of the second element. A person skilled in the art of metal/semiconductor junctions can readily select materials for first and second elements 12 and 14, and assemble them to form a junction 13 according to the invention.

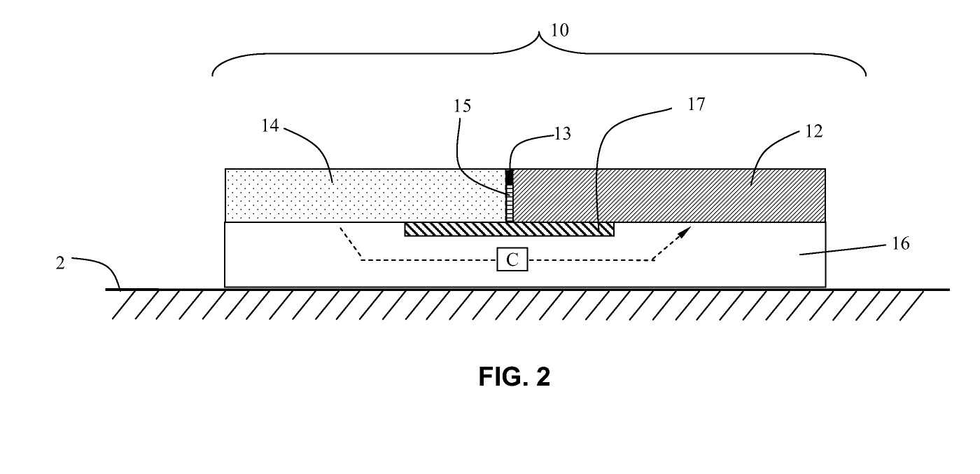

To reduce the area of contact between the first element and the second element, a vertical insulator 15 may be interposed between the first element and the second element to reduce the size of the junction 13 (see FIG. 2). This will increase the first electrical resistance of the junction 13. The insulator 15 may occupy up to ⅔ of the connection area between the first element and the second element, thus reducing the size of junction 13 by ⅔.

The third element 16 may comprise any suitable electrically conductive medium with a second electrical resistance that is significantly less than the first electrical resistance of the junction 13. Ideally, the electrically conductive material or materials of the third element 16 will have sufficient conductivity and a second electrical resistance sufficiently less than the first electrical resistance of the junction 13 to permit an electrical current with a current density or intensity preferably ranging from about 1 μA to about 1000 μA, more preferably from about 5 μA to about 800 μA, and most preferably from about 300 μA to about 700 μA. Preferably, the electrically conductive medium in the third element is selected from the group consisting of: (1) electrolyte solutions; (2) hydrogels; (3) conductive adhesives; and (4) combinations thereof. As long as the third element 16 exhibits sufficiently high electrical conductivity and significantly less electrical resistance than junction 13, the desired cross-flow microcurrent over the skin surface will be generated in the absence of a power source. Preferably, the electrically conductive medium as employed in the system of the present invention is selected from the group consisting of: (1) electrolyte solutions containing inert electrolytes, such as sodium chloride, potassium chloride, and the like; (2) hydrogels, such as those typically used for forming medical electrodes (e.g., those composed of crosslinked polymers like polyethylene oxide, poly(2-acrylamido-2-methyl-1-propanesulfonic acid) or poly AMPS, and polyvinylpyrrolidone); (3) conductive adhesives, such as glycidyl ether epoxy resin, phenoxy resins, and the like; and (4) combinations thereof.

Hydrogel is a particularly preferred electrically conductive medium for practice of the present invention. Hydrogel comprises a network of polymer chains that are water-insoluble and dispersed in an aqueous medium, thereby forming a colloidal gel. Hydrogel can be formed of synthetic polymers, such as crosslinked polyethylene oxide, crosslinked poly(2-acrylamido-2-methyl-1-propanesulfonic acid) or poly AMPS, crosslinked polyvinylpyrrolidone, polyvinyl alcohol, sodium polyacrylates, acrylate polymers and copolymers with abundant hydrophilic groups, as well as natural polymers, such as agarose, methylcellulose, hylaronan, and the like. Correspondingly, the third element 16 may consist essentially of a hydrogel which has sufficient adhesiveness and can be readily applied to a skin surface and stay thereon. Element 16 may also comprise hydrogel saturated with an aqueous electrolyte solution. The third element 16 may also simply comprise an aqueous solution of an electrolyte such as sodium chloride or potassium chloride, which can be directly applied to a skin surface, with subsequent application of the first and second elements, either individually or as a pre-assembled strip or sheet. Further, the third element 16 may include a non-conductive substrate layer (not shown) with a conductive adhesive coating. The arrangement and construction of element 16 can be readily modified by a person of ordinary skill in the art according to specific requirements for the system 10, and the scope of the present invention is thus not limited to any specific arrangement and construction described hereinabove.

In a preferred embodiment of the present invention, the first element comprises one or more elemental metals that can act as the electron donor or acceptor. For example, the first element may comprise one or more elemental metals selected from the group consisting of gold, rhodium, platinum, iridium, silver, copper, aluminum and alloys and mixtures thereof. The first element can take the form of a solid block, a sheet, a woven or non-woven fabric, or any other suitable form. If more than one elemental metal is used, the metals may be laminated, coated, tinned or otherwise formed.

The second element comprises one or more semiconductor materials selected from the group consisting of zinc oxide, tin oxide, iridium oxide, indium oxide, aluminum oxide, and mixtures thereof. The second element may be provided in any form suitable for semiconductor construction. Preferably, the semiconductor material in the second element is substantially free of dopant.

The topical or cosmetic system may be in the form of a device such as a patch for treating skin. In the device, the third element 16 in addition to being an electrically conductive material comprises a substrate patch having at least one side with sufficient adhesiveness for application to a skin surface. Supported on the third element 16 is the first element 12 comprising at least one elemental metal capable of acting as an electron donor or acceptor and the second element 14 comprising at least one semiconductor material capable of acting as an electron acceptor or donor. The first and second elements 12, 14 are in contact with each other through a metal/semiconductor junction 13 having a first electrical resistance. The substrate patch (third element 16) comprises an electrically conductive medium that extends across the skin surface and electrically connects the first and second elements. The electrically conductive medium of the substrate patch (third element 16) has a second electrical resistance. The second electrical resistance of the substrate patch (third element 16) is significantly less than the first electrical resistance of junction 13, so that an electrical current is generated between the first and second elements 12, 14 that flows across the skin surface through the conductive medium of the substrate patch in the absence of any power source.

The invention further comprises a method for treating skin, comprising generating an electric current that flows across a skin surface by using the system and/or device described above. The invention can be used to treat skin for conditions selected from skin wounds, acute or chronic photo-damage, chemical or environmental damage, wrinkles, fine lines, reduced elasticity and reduced collagen or water content caused by skin aging.

The topical or cosmetic system of the present invention is preferably embodied in a patch-like cosmetic article or device with relatively simple structure and fewer components, in comparison with conventional dermal patches typically used for transdermal delivery of active cosmetic or pharmaceutical ingredients into the skin. Such a patch-like cosmetic device can be easily manufactured at relatively low costs and readily applied to a skin surface for treatment thereof with few pre-treatment or preparation steps. For example, the present invention may provide a topical or cosmetic patch that includes a substrate patch with at least one side having sufficient adhesiveness for application to a skin surface. A first region (comparable to the first element 12) of this substrate patch includes at least one component capable of acting as an electron donor or acceptor, and a second region (comparable to the second element 14) of this patch includes at least one component capable of acting as an electron acceptor or donor. The first and second regions are in contact with each other through a metal/semiconductor junction having a first electrical resistance. The substrate patch further includes an electrically conductive medium, which electrically connects the electron donor component, e.g., at the first region, with the electron acceptor component, e.g., at the second region, thereby generating an electric current that flows through the substrate patch across the skin surface in the absence of any power source. The composition and construction of the substrate may be similar to the element 16 in FIG. 1, and the compositions and constructions of the first and second regions may be similar to the elements 12 and 14 in FIG. 1, as described hereinabove.

Note that the third element 16 (the substrate patch) of the present invention may comprise additional layers of conductive and non-conductive materials, as long as the first and second elements 12, 14 are in direct contact with each other through junction 13 and with at least some conductive material in the third element 16. For example, to increase the distance that the electric current C will travel through the conductive medium and correspondingly across the skin, an horizontal insulator 17 (FIG. 2) may interposed between a middle portion of the third element 16 and adjacent portions of the elements 12 and 14 at the junction 13.

The third element 16 is to be applied directly onto a skin surface for generation of the cross-flow micro-electric current across or adjacent to the skin surface as described above. For transportation and storage purposes, such conductive layer may be covered by a removable non-conductive protective layer (not shown), and prior to application to the skin surface, such protective layer is removed so as to allow the third element 16 to directly contact the skin surface.

In order to provide a microcurrent-generating cosmetic patch with extended shelf life, it may be desirable to use a material with a variable electrical conductivity in the substrate patch. Specifically, the material is initially provided in a non-conductive or a less conductive phase, so that little or no electrical current is generated in the substrate patch and the substrate patch can be stored for an extended period of time prior to use. At the time of actual use, the material can be converted to a conductive or a more conductive phase via one or more simple treatment steps, so that an electrical current of desired intensity can be generated in the substrate patch for skin treatment. For example, the substrate patch may be initially formed of dried hydrogel, which has little or no electrical conductivity, and at the time of use, the user may wet the dried hydrogel patch with simply water or an aqueous wetting solution that contains additional electrolytes, to form a conductive hydrogel patch that is capable of generating a microcurrent across the skin surface.

Alternatively, the substrate patch and the assembly has first and second elements with a metal/semiconductor junction that is reduced by up to ⅔ in contact area by a vertical electrical insulator 15 which covers ⅔ of the contact area between the elements which are fabricated separately and assembled prior to use.

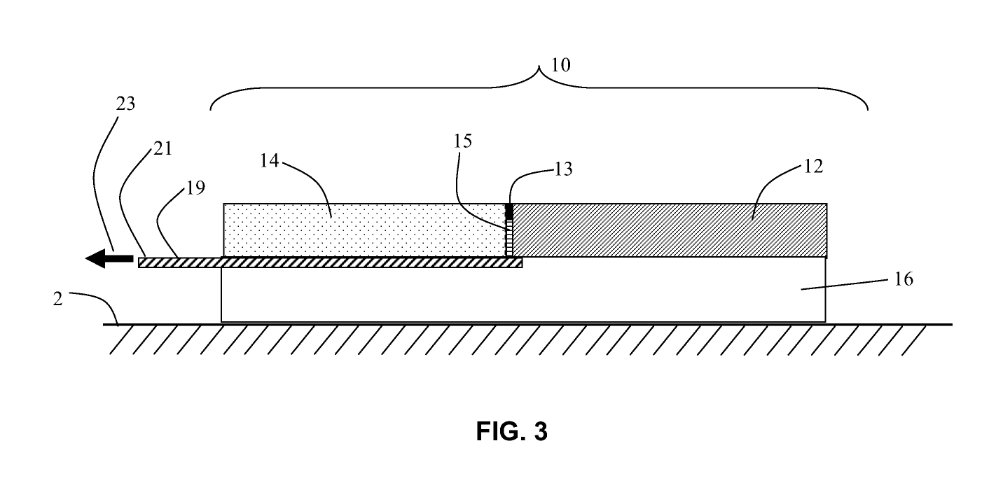

Alternatively, a temporary, removable insulator 19 (FIG. 3) in the form of a non-conductive sheet may be provided between the third element 16 and at least one of the first element 12 or second element 14 to prevent current from flowing through the third element 16 until desired. Prior to use, the temporary insulator would be removed by pulling on free end 21 in the direction of arrow 23, such that both the first element 12 and second element 14 can contact the third element 16 to start the flow of current through the third element 16.

The third element 16 may also include topical or cosmetic compositions formulated with pharmaceutically or cosmetically acceptable carriers. The term “pharmaceutically or cosmetically acceptable carrier” refers to a carrier, for either pharmaceutical or cosmetic use, which carrier delivers the active components to the intended target and which will not cause harm to humans or other recipient organisms. As used herein, “pharmaceutical” or “cosmetic” will be understood to encompass pharmaceuticals or cosmetics for both humans and animals. The carrier can be provided in any form convenient for topical application to the skin. Such forms include, but are not limited to gels, creams, dispersions, emulsions (water-in-oil or oil-in-water), suspensions, lotions, foams, mousses and the like. By delivering the topical or cosmetic formulas from the third element 16 to the skin of the user, the patch of the present invention would thus provide electrical current therapy as well as topical or cosmetic composition therapy (e.g., moisturizing, hydrating, etc.).

The topical or cosmetic compositions may also have incorporated active skin care agents which are used for skin treatment, or which are routinely applied topically. Examples of such active skin care agents which may form part of the above-described compositions include, but are not limited to, those that improve or eradicate age spots, keratoses and wrinkles, analgesics, anesthetics, anti-acne agents, antibacterials, antiyeast agents, antifungal agents, antiviral agents, antidandruff agents, antidermatitis agents, antipruritic agents, antiemetics, antimotion sickness agents, anti-inflammatory agents, antihyperkeratolytic agents, anti-dry skin agents, antiperspirants, antipsoriatic agents, antiseborrheic agents, hair conditioners and hair treatment agents, antiaging agents, antiwrinkle agents, antiasthmatic agents and bronchodilators, sunscreen agents, antihistamine agents, skin lightening agents, depigmenting agents, wound-healing agents, vitamins, corticosteroids, tanning agents, sunscreens or hormones. More specific examples of useful active skin care agents include retinoids, topical cardiovascular agents, clotrimazole, ketoconazole, miconozole, griseofulvin, hydroxyzine, diphenhydramine, pramoxine, lidocaine, procaine, mepivacaine, monobenzone, erythromycin, tetracycline, clindamycin, meclocyline, hydroquinone, minocycline, naproxen, ibuprofen, theophylline, cromolyn, albuterol, retinol, retinoic acid, 13-cis retinoic acid, hydrocortisone, hydrocortisone 21 acetate, hydrocortisone 17-valerate, hydrocortisone 17-butyrate, betamethasone valerate, betamethasone diproprionate, triamcinolone acetonide, fluocinonide, clobetasol, proprionate, benzoyl peroxide, crotamiton, propranolol, promethazine, vitamin A palmitate, vitamin E acetate, DHEA and derivatives thereof, alpha- or beta-hydroxy acids, and mixtures thereof. The amount of active skin care agent to be used in any given composition is readily determined in accordance with its usual dosage. In adding of such further components to the microcurrent-generating system of the present invention, however, consideration should be given to the standard oxidation potentials of the additional components, so that the additional components do not interfere with the intended interactions between the respective elements of the system.

The topical or cosmetic compositions may further comprise other components which may be chosen depending on the carrier and/or the intended use of the compositions. Additional components include, but are not limited to: water soluble colorants (such as FD&C Blue #1); oil soluble colorants (such as D&C Green #6); water soluble sunscreens (such as Eusolex 232); oil soluble sunscreens (such as Octyl Methoxycinnamate); particulate sunscreens (such as Zinc Oxide); antioxidants (such as BHT); chelating agents (such as Disodium EDTA); emulsion stabilizers (such as carbomer); preservatives (such as Methyl Paraben); fragrances (such as pinene); flavoring agents (such as sorbitol); humectants (such as glycerine); waterproofing agents (such as PVP/Eicosene Copolymer); water soluble film-formers (such as Hydroxypropyl methylcellulose); oil-soluble film formers (such as Hydrogenated C-9 Resin); cationic polymers (such as Polyquaternium 10); anionic polymers (such as xanthan gum); vitamins (such as Tocopherol); and the like.

The microcurrent-generating system of the present invention can be used in a number of different therapeutic or preventive applications. In general terms, since the presence of an electric potential at the skin surface is shown to be characteristic of normal, healthy skin, application of the microcurrent-generating system of the present invention can be employed, for example, as a regularly applied preventative treatment of skin damage, e.g. redness and irritation commonly associated with dry skin or exposure to sun, heat and/or cold, and to promote and maintain overall skin health. It can also serve as a spot treatment to reduce the effects of inflammation or irritation on an already damaged skin surface, wherein the treatment can be applied and repeated, as needed. In this regard, the electrically conductive medium may be directly mixed with other skin care actives for use in treatment of skin conditions, as described above. However, the system of the present invention can also be used alone, i.e., without any skin care additives, for reduction of fine lines, wrinkles, acute or chronic skin damages caused by sun exposure or environmental assaults and also for reduction of irritation and inflammation associated with dry skin, severe dry skin, dandruff, acne, keratoses, psoriasis, eczema, skin flakiness, pruritus, lentigines, melasmas, warts, blemished skin, hyperpigmented skin, hyperkeratotic skin, or inflammatory dermatoses, which conditions may or may not also be treated with a skin active agent. In addition, the microcurrent-generating system or device of the present invention can be used as an adjunct to the wound healing process. As shown above, healing skin is known to be associated with a measurable, increased current. The system and device of the present invention can be used to enhance the naturally occurring process, either by direct combination with wound-healing active agents, or alone in a separate application.

It will be understood by those skilled in the art that the phrase “treatment or prevention of skin damage” as used in the present specification encompasses each of the enumerated specific applications, as well as any not specifically enumerated expressly herein. In particular, it will be understood that “prevention of skin damage” is meant to include routine maintenance of skin health without reference to prevention of a specific skin condition, as well as referring to prevention of specific conditions or problems.

While the invention has been described herein with reference to specific aspects, features, and embodiments, it will be recognized that the invention is not thus limited, but rather extends to and encompasses other variations, modifications and alternative embodiments. Accordingly, the invention is intended to be broadly interpreted and construed to encompass all such other variations, modifications, and alternative embodiments, as being within the scope and spirit of the invention as hereinafter claimed.

What we claim is:

1. A topical or cosmetic system comprising:
(a) a first element comprising at least one elemental metal;
(b) a second element comprising at least one semiconductor material, wherein said first and second element are in contact with each other through a metal/semiconductor junction having a first electrical resistance; and
(c) a third element extending across the skin surface, wherein said third element comprises an electrically conductive medium for electrically connects said first and second elements, said electrically conductive medium has a second electrical resistance,
wherein the second electrical resistance is less than the first electrical resistance, such that an electrical current is generated between the first element and second element that flows across the skin surface through said conductive medium in the absence of any power source.

2. The system of claim 1, wherein the second electrical resistance is less than half of the first electrical resistance.

3. The system of claim 1, wherein the second electrical resistance is less than 10% of the first electrical resistance.

4. The system of claim 1, wherein the second electrical resistance is less than 1% of the first electrical resistance.

5. The system of claim 1, where the electrically conductive medium has a sufficiently high conductivity so that the electrical current so generated has a current density ranging from about 1 μA to about 1000 μA.

6. The system of claim 1, wherein the first element comprises one or more elemental metals selected from the group consisting of gold, rhodium, platinum, iridium, silver, copper, aluminum, and alloys and mixtures thereof.

7. The system of claim 1, wherein the second element comprises one or more semiconductor materials selected from the group consisting of zinc oxide, tin oxide, iridium oxide, indium oxide, aluminum oxide, and mixtures thereof.

8. The system of claim 1, wherein the semiconductor material in the second element is substantially free of dopant.

9. The system of claim 1, wherein the electrically conductive medium in the third element is selected from the group consisting of: (1) electrolyte solutions; (2) hydrogels; (3) conductive adhesives; and (4) combinations thereof.

10. The system of claim 9, wherein the electrically conductive medium comprises hydrogel.

11. A topical or cosmetic device comprising a substrate patch having at least one side with sufficient adhesiveness for application to a skin surface, wherein a first region of said substrate patch comprises at least one elemental metal, wherein a second region of said substrate patch comprises at least one semiconductor material, said first and second regions are in contact with each other through a metal/semiconductor junction having a first electrical resistance, and wherein said substrate patch comprises an electrically conductive medium that extends across the skin surface and electrically connects the first and second regions, said electrically conductive medium has a second electrical resistance, wherein the second electrical resistance is less than the first electrical resistance, such that an electrical current is generated between the first and second region that flows across the skin surface through said conductive medium in the absence of any power source.

12. A method for preventing or treating skin damage, comprising generating an electric current that flows across a skin surface by using the system of claim 1.

13. The method of claim 12, wherein the skin damage are selected from the group consisting of skin wounds, acute or chronic photo-damages, chemical or environmental damages, wrinkles, fine lines, reduced elasticity and reduced collagen or water content caused by skin aging.

14. The topical or cosmetic system of claim 1 further comprising a vertical insulator interposed between the first element and the second element to reduce the size of the metal/semiconductor junction between the first element and the second element.

15. The topical or cosmetic system of claim 14 wherein the vertical insulator reduces the metal/semiconductor junction in size by up to ⅔.

16. The topical or cosmetic system of claim 1 further comprising a horizontal insulator interposed between a middle portion of the third element 16 and adjacent portions of the elements 12 and 14 at the junction 13.

17. The topical or cosmetic system of claim 1 further comprising a selectively removable insulator in the form of a non-conductive sheet interposed between the third element and at least one of the first element and the second element such that current is prevented from flowing through the third element 16 until the removable insulator is removed.
